(19) United States
(12) Patent Application Publication
(10) Pub. No.: US 2020/0146578 A1
(43) Pub. Date: May 14, 2020

(54) DEFORMABLE HIGH-DENSITY AND PRECISION BALLOON CARDIAC MAPPING CATHETER

(71) Applicant: Wuxi People's Hospital, Jiangsu (CN)

(72) Inventors: Shipeng Dang, Jiangsu (CN); Ruxing Wang, Jiangsu (CN); Changying Zhang, Jiangsu (CN); Kulin Li, Jiangsu (CN); Jie Zheng, Jiangsu (CN); Xiaoyu Liu, Jiangsu (CN)

(73) Assignee: Wuxi People's Hospital, Jiangsu (CN)

(21) Appl. No.: 16/671,167

(22) Filed: Nov. 1, 2019

(30) Foreign Application Priority Data
Nov. 8, 2018 (CN) 201811326524.4

(51) Int. Cl. A61B 5/042; A61B 5/00; A61M 25/10

(52) U.S. Cl. CPC A61B 5/0422; A61B 2562/028; A61M 25/10182; A61B 5/6853

(57) ABSTRACT

A deformable high-density and precision balloon cardiac mapping catheter includes a catheter, an electrode head mounted at one end of the catheter, and a handle mounted at the other end of the catheter. A head electrode pair, a middle expansion body and a rear expansion body are sequentially disposed on the electrode head from left to right. A liquid storage apparatus is disposed on a surface of the handle. The liquid storage apparatus includes a reservoir tube. A liquid outlet is disposed at one end of the reservoir tube.

### CROSS-REFERENCE TO RELATED APPLICATION

This application claims the priority benefit of China application serial no. 201811326524.4, filed on Nov. 8, 2018. The entirety of the above-mentioned patent application is hereby incorporated by reference herein and made a part of this specification.

### BACKGROUND

### Technical Field

The present invention relates to the field of medical device technologies, and specifically, to a deformable high-density and precision balloon cardiac mapping catheter.

### Description of Related Art

A cardiac mapping catheter is used to construct a three-dimensional (3D) electroanatomical model of a cardiac chamber. For various types of cardiac arrhythmias, especially complicated cardiac arrhythmias, it is vital to learn about the electrical conduction information and anatomical information at various locations in the cardiac chamber to understand the mechanism of the cardiac arrhythmias. For example, the electroanatomical model of the left atrium needs to be constructed during pulmonary vein isolation for treating atrial fibrillation. If the precise electroanatomical model of the pulmonary vein antrum can be constructed to learn about the orientations and lengths of the muscle bundle of the pulmonary veins, the efficiency of the pulmonary vein isolation can be improved.

At present, contact mapping catheters for cardiac chamber mapping mainly include 4 to 10-pole linear mapping catheters, 10 to 12-pole circular mapping catheters, a 16-pole grid mapping catheter, and a 64-pole basket mapping catheter. These catheters have a limited quantity of electrodes in contact with the cardiac chamber and are sometimes susceptible to poor contact or even push the heart to produce imprecise cardiac electrical information and anatomical positioning, and as a result the established 3D cardiac chamber model is inaccurate. In view of this, we propose a deformable high-density and precision balloon cardiac mapping catheter.

### SUMMARY

The objective of the present invention is to provide a deformable high-density and precision balloon cardiac mapping catheter, to resolve the problems in the background art that the mapping catheters have a limited quantity of electrodes in contact with the heart and are sometimes susceptible to poor contact.

To achieve the foregoing objective, the present invention provides the following technical solution:

A deformable high-density and precision balloon cardiac mapping catheter includes a catheter, an electrode head mounted at one end of the catheter, and a handle mounted at the other end of the catheter, where a tail wire interface is disposed at a bottom of the handle, a head electrode pair, a middle expansion body and a rear expansion body are sequentially disposed on the electrode head from left to right, a plurality of first electrode pairs are disposed on a side, near the middle expansion body and the rear expansion body of the electrode head, a magnetic sensor is disposed at a center of a surface of the head electrode pair, and a plurality of second electrode pairs are annularly arrayed on an outer side of the magnetic sensor.

Preferably, each of the first electrode pairs and the second electrode pairs is formed by two microelectrodes, and each microelectrode is connected to a side of the tail wire interface by a wire.

Preferably, the head electrode pair and the middle expansion body have an integral structure.

Preferably, the electrode head has a hollow structure, and the electrode head is in communication with the catheter.

Preferably, a liquid storage apparatus is disposed on a surface of the handle, the liquid storage apparatus includes a reservoir tube, a liquid outlet is disposed at one end of the reservoir tube, a sealed tube inserted in the reservoir tube is disposed at an end of the liquid outlet, a push rod is disposed at the other end of the reservoir tube, a plug plate slidably connected to the reservoir tube is disposed at one end of the push rod, and a push plate is mounted at the other end of the push rod.

Preferably, the push plate and the push rod have an integral structure.

Compared with the prior art, the beneficial effects of the present invention are as follows.

1. In the deformable high-density and precision balloon cardiac mapping catheter, the liquid storage apparatus is disposed to inject a liquid into the electrode head, and the head electrode pair and the middle expansion body are expanded to enable the electrode head to form a rod shape, a conical shape or a spherical shape. These shapes have adjustable lengths and diameters. Rod-shaped and conical-shaped parts may enter the positions such as the coronary veins, the superior and inferior venae cavae, the pulmonary veins, and the left and right ventricular outflow tracts, to perform fast and precise electroanatomical mapping of these locations.

2. In the deformable high-density and precision balloon cardiac mapping catheter, the rod-shaped and conical-shaped part of the electrode head are bendable to form an arc shape, and a catheter connected to the electrode head is bidirectionally bendable, so that the electrode head can move inside the cardiac chamber and stick to the endocardium, to implement electroanatomical mapping of the cardiac chamber.

3. In the deformable high-density and precision balloon cardiac mapping catheter, the head of the catheter is a balloon filled with a gas or liquid and does not push the heart to deform the cardiac chamber. The surface of the head is filled with electrodes, and the head may perfectly stick to various positions of the heart, thereby facilitating fast and high-density and precision electroanatomical mapping of the cardiac chamber.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic diagram when a balloon electrode head is not expanded according to the present invention;

FIG. 2 is a schematic diagram when the balloon electrode head is partially expanded according to the present invention;

FIG. 3 is a schematic structural diagram of an electrode head according to the present invention;

FIG. 4 is a schematic structural diagram of a head electrode pair according to the present invention;

FIG. 5 is a schematic structural exploded diagram of a liquid storage apparatus according to the present invention;

FIG. 6 is a schematic structural diagram of an electrode head according to specific Embodiment 4 of the present invention; and

FIG. 7 is a schematic structural diagram of an electrode head according to specific Embodiment 5 of the present invention.

### DESCRIPTION OF THE EMBODIMENTS

The technical solutions according to the embodiments of the present invention are clearly and thoroughly described with reference to the accompanying drawings in the embodiments of the present invention. The described embodiments are merely exemplary ones, but are not all the embodiments of the present invention. Based on the embodiments of the present invention, all other embodiments derived by persons of ordinary skill in the art without any creative efforts shall fall within the protection scope of the present invention.

In the description of the present invention, it needs to be understood that orientation or location relationships indicated by terms “center”, “longitudinal”, “transverse”, “length”, “width”, “thickness”, “up”, “down”, “front”, “rear”, “left”, “right”, “vertical”, “horizontal”, “top”, “bottom”, “inside”, “outside”, “clockwise”, and “counterclockwise” are based on orientation or location relationships shown in the accompanying drawings, and are only used to facilitate description of the present invention and simplify description, but are not used to indicate or imply that the devices or elements must have specific orientations or are constructed and operated by using specific orientations, and therefore, cannot be understood as a limitation to the present invention.

In addition, the terms “first” and “second” are only used for description, but cannot be used to indicate or imply relative importance or implicitly specify the number of indicated technical features. Therefore, one or more features may be explicitly or implicitly included when such features are defined by “first” and “second”. In the description of the present invention, “a plurality of” means two or more than two, unless otherwise explicitly or particularly defined.

### Embodiment 1

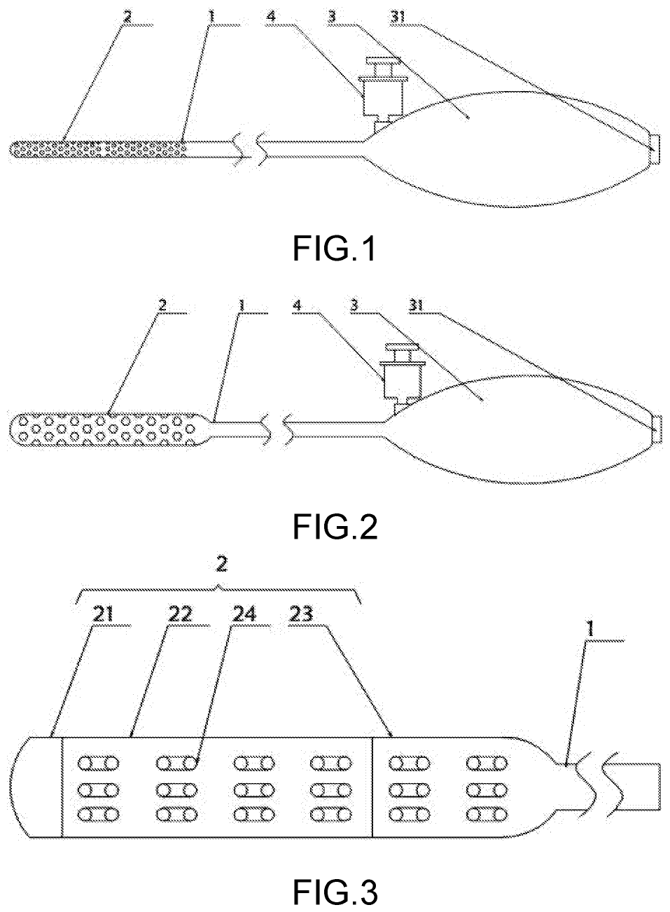

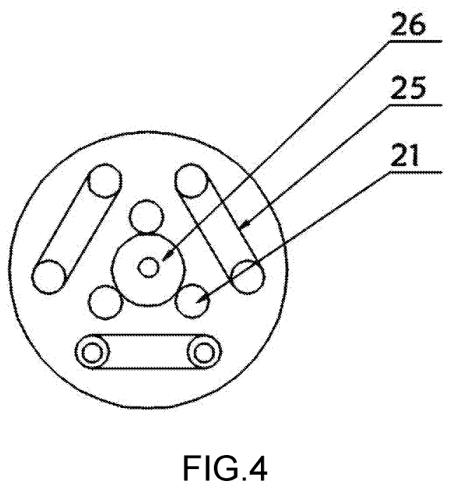

As shown in FIG. 1 to FIG. 4, a deformable high-density and precision balloon cardiac mapping catheter includes a catheter 1, an electrode head 2 mounted at one end of the catheter 1, and a handle 3 mounted at the other end of the catheter 1. A tail wire interface 31 is disposed at a bottom of the handle 3. A head electrode pair 21, a middle expansion body 22 and a rear expansion body 23 are sequentially disposed on the electrode head 2 from left to right. A plurality of first electrode pairs 24 are disposed on a side, near the middle expansion body 22 and the rear expansion body 23 of the electrode head 2. A magnetic sensor 26 is disposed at a center of a surface of the head electrode pair 21. A plurality of second electrode pairs 25 are annularly arrayed on an outer side of the magnetic sensor 26. Each of the first electrode pairs 24 and the second electrode pairs 25 is formed by two microelectrodes. Each microelectrode is connected to a side of the tail wire interface 31 by a wire. The head electrode pair 21 and the middle expansion body 22 have an integral structure. The electrode head 2 has a hollow structure. The electrode head 2 is in communication with the catheter 1.

In this embodiment, two electrodes are bonded by insulating rubber to form one electrode pair, and two adjacent electrode pairs are bonded and fixed by elastic rubber, so that when the electrode head is expanded, the gap between electrodes is kept unchanged, and the gap between electrode pairs can change.

Specifically, the head electrode pair 21, the middle expansion body 22, and the rear expansion body 23 are all made of an elastic rubber material. A thickness of the head electrode pair 21 is greater than that of the middle expansion body 22, and a thickness of the middle expansion body 22 is greater than that of the rear expansion body 23. The deformation effects of the head electrode pair 21, the middle expansion body 22 and the rear expansion body 23 are controlled by using the different thicknesses of the head electrode pair 21, the middle expansion body 22, and the rear expansion body 23.

In addition, the catheter 1 is a PP foldable tube, so that the catheter 1 may be folded bidirectionally, thereby facilitating the use of the catheter 1.

In addition, the catheter 1 and the electrode head 2 are bonded and fixed by a glue, so that the electrode head is conveniently mounted on a surface of the catheter 1.

It should be noted that the magnetic sensor 26 is a magnetic coil. A magnetic field generator is disposed outside the magnetic sensor 26. The magnetic field generator emits a plurality of magnetic fields, so that each magnetic field can generate an induced current in the magnetic sensor 26.

In this embodiment, 81 electrodes are arranged outside the middle expansion body 22 and the rear expansion body 23.

### Embodiment 2

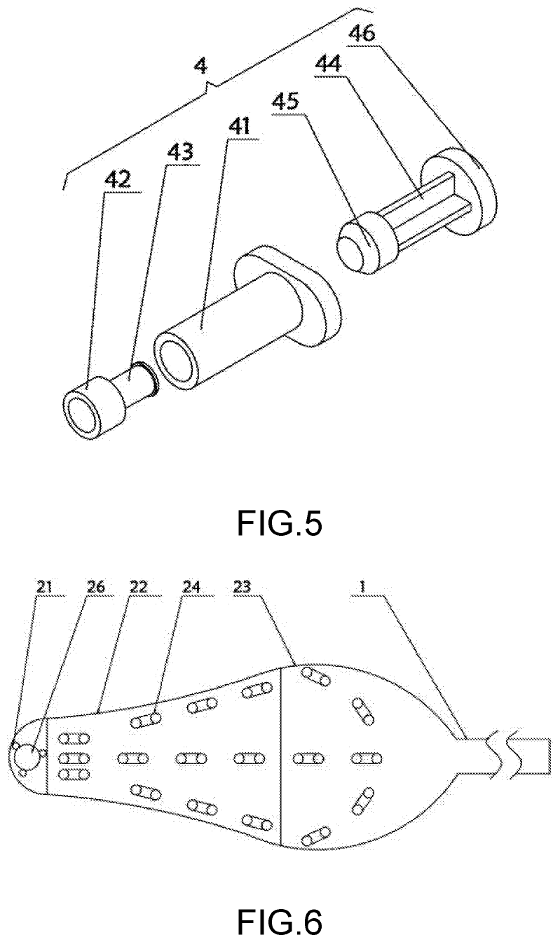

As shown in FIG. 5, a liquid storage apparatus 4 is disposed on a surface of the handle 3. The liquid storage apparatus 4 includes a reservoir tube 41. A liquid outlet 42 is disposed at one end of the reservoir tube 41. A sealed tube 43 inserted in the reservoir tube 41 is disposed at an end of the liquid outlet 42. A push rod 44 is disposed at the other end of the reservoir tube 41. A plug plate 45 slidably connected to the reservoir tube 41 is disposed at one end of the push rod 44. A push plate 46 is mounted at the other end of the push rod 44. The push plate 46 and the push rod 44 have an integral structure.

In this embodiment, the liquid outlet 42 penetrates the surface of the handle 3 and is connected by a sealant to a connection between the liquid outlet 42 and the handle 3, to facilitate the entry of a liquid from the liquid outlet 42 into the handle 3.

Specifically, an annular sealing ring should further be disposed at one end of the sealed tube 43, so that the sealed tube 43 and the reservoir tube 41 are tightly connected.

In addition, the plug plate 45 is made of a rubber material, so that the plug plate 45 tightly fits the inner wall of the reservoir tube 41 and the plug plate 45 is used to push the liquid from inside the reservoir tube 41.

### Embodiment 3

The rear expansion body 23 of the electrode head 2 is pushed into the catheter 1, and the push plate 46 is pressed to enable the push rod 44 to slide inside the reservoir tube 41, so that some liquid inside the reservoir tube 41 is pressed into the catheter 1 and enters the electrode head 2. In this case, the rear expansion body 23 is located inside the catheter 1 and cannot be expanded. The middle expansion body 22 is pressed by the liquid and expanded to form the shape shown in FIG. 2. In this case, the catheter 1 is inserted into the heart, and the electrode head 2 is bent. The electrode head 2 is rotated to enable the first electrode pairs 24 and the second electrode pairs 25 to be in full contact with the wall of the heart and at the same time enter the positions such as the pulmonary veins, the superior vena cava, and the ventricular outflow tracts to rapidly establish accurate models of the positions.

### Embodiment 4

The electrode head 2 is completely located outside the catheter 1, and the push plate 46 is pressed to enable the push rod 44 to slide inside the reservoir tube 41. Some liquid inside the reservoir tube 41 is pressed into the catheter 1 and enters the electrode head 2, and the rear expansion body 23 is expanded incompletely. In this case, because the thickness of the middle expansion body 22 is greater than that of the rear expansion body 23, the expansion effect of the middle expansion body 22 is weaker than the rear expansion body 23, to form the shape in FIG. 6.

### Embodiment 5

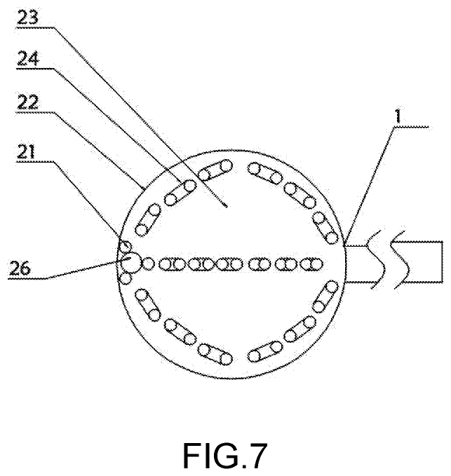

The electrode head 2 is completely located outside the catheter 1, and the push plate 46 is pressed to enable the push rod 44 to slide inside the reservoir tube 41. All the liquid inside the reservoir tube 41 is pressed into the catheter 1 and enters the electrode head 2, and the middle expansion body 22 and the rear expansion body 23 are completely expanded, to form the shape in FIG. 7.

In the foregoing embodiments, the liquid storage apparatus 4 is disposed to inject a liquid into the electrode head 2, and the head electrode pair 21, the middle expansion body 22, and the rear expansion body 23 are expanded to change the overall shape of the electrode head 2 and achieve perfect fit at various positions of the heart without pushing the heart and without needing to repeatedly steer in the cardiac chamber, thereby facilitating high-density and precision mapping.

The wall thicknesses of the middle expansion body 22 and the rear expansion body 23 gradually change. During actual manufacturing, a small gap may be opened first, and after an electrode pair is then inserted, the head electrode pair 21 and the middle expansion body 22 are sealed. An electrode wire runs inside the middle expansion body 22 to be connected to a signal acquisition end.

A balloon may be filled with either a gas or liquid. An apparatus for controlling filling and releasing is not protected by the patent and may be implemented by using the prior art. The size of the balloon may be better controlled by filling or releasing a liquid, because a gas can be compressed. However, both the filling of a gas and the filling of a liquid should be protected. A reservoir tank may be manually operated to control the size of a balloon. The volume of the reservoir tank may be 30 milliliters to 50 milliliters and does not need to be too large. However, a valve needs to be used in combination to prevent liquid backflow.

The basic principles and main features of the present invention and the advantages of the present invention are shown and described above. A person skilled in the art should understand that the present invention is not limited to the foregoing embodiments. Only preferred examples of the present invention are described in the foregoing embodiments and specification, but are not used to limit the present invention. Various changes and improvements may be made to the present invention without departing from the spirit and scope of the present invention. These changes and improvements all fall within the protection scope of the present invention. The protection scope of the present invention is defined by the appended claims and equivalents thereof.

What is claimed is:

1. A deformable high-density and precision balloon cardiac mapping catheter, comprising a catheter, an electrode head mounted at one end of the catheter, and a handle mounted at the other end of the catheter, wherein a tail wire interface is disposed at a bottom of the handle, a head electrode pair, a middle expansion body and a rear expansion body are sequentially disposed on the electrode head from left to right, a plurality of first electrode pairs are disposed on a side, near the middle expansion body and the rear expansion body of the electrode head, a magnetic sensor is disposed at a center of a surface of the head electrode pair, and a plurality of second electrode pairs are annularly arrayed on an outer side of the magnetic sensor.

2. The deformable high-density and precision balloon cardiac mapping catheter according to claim 1, wherein each of the first electrode pairs and the second electrode pairs is formed by two microelectrodes, and each microelectrode is connected to a side of the tail wire interface by a wire.

3. The deformable high-density and precision balloon cardiac mapping catheter according to claim 1, wherein the head electrode pair and the middle expansion body have an integral structure.

4. The deformable high-density and precision balloon cardiac mapping catheter according to claim 1, wherein the electrode head has a hollow structure, and the electrode head is in communication with the catheter.

5. The deformable high-density and precision balloon cardiac mapping catheter according to claim 1, wherein a liquid storage apparatus is disposed on a surface of the handle, the liquid storage apparatus comprises a reservoir tube, a liquid outlet is disposed at one end of the reservoir tube, a sealed tube inserted in the reservoir tube is disposed at an end of the liquid outlet, a push rod is disposed at the other end of the reservoir tube, a plug plate slidably connected to the reservoir tube is disposed at one end of the push rod, and a push plate is mounted at the other end of the push rod.

6. The deformable high-density and precision balloon cardiac mapping catheter according to claim 5, wherein the push plate and the push rod have an integral structure.
